(19) United States
(12) Patent Application Publication
(10) Pub. No.: US 2021/0008381 A1
(43) Pub. Date: Jan. 14, 2021

(54) COMPACT PORTABLE ELECTROMAGNETIC FIELD AND ION EMITTER APPARATUS

(71) Applicant: Yat Fai Wong, Vancouver (CA)

(72) Inventor: Yat Fai Wong, Vancouver (CA)

(73) Assignee: Yat Fai Wong, Vancouver (CA)

(21) Appl. No.: 17/033,852

(22) Filed: Sep. 27, 2020

(51) Int. Cl. A61N 2/00; A61N 2/02

(52) U.S. Cl. CPC A61N 2/002; A61N 2/008; A61N 2/02

(57) ABSTRACT

The present invention relates to a portable device capable of being operated safely and effectively by patients which produces a time varying, magnetic field having a low frequency and low flux density effective in the treatment of a wide variety of physical and mental disorders. More specifically, it relates to a method and system for generating electromagnetic fields from amplified, digital frequencies from a mobile computing device. In an aspect, a portable electromagnetic energy generating apparatus is disclosed. The apparatus includes a connecting means for connecting the portable electromagnetic energy generating apparatus with one or more electric signal generating means to receive the electric signal from the one or more electric signal generating means at least in a wired communication mode or in a wireless communication mode; an electromagnetic energy generating means to produce one or more electromagnetic fields on receipt of the electric signal generated by the one or more electric signal generating means, wherein the one or more produced electromagnetic fields are associated with the received electric signal; and at least a crystal or a gem to generate one or more ions in presence of the one or more produced electromagnetic fields

### TECHNICAL FIELD

The present invention generally relates to resonant frequencies. More particularly, the present invention relates to a portable device capable of being operated safely and effectively by patients which produces a time varying, magnetic field having a low frequency and low flux density effective in the treatment of a wide variety of physical and mental disorders. More specifically, it relates to a method and system for generating electromagnetic fields from amplified, digital frequencies from a mobile computing device.

### BACKGROUND

Magnetic fields have long been used for the treatment of physical injuries and chronic pain. Early magnetic therapy involved the use of static magnetic fields produced by permanent magnets incorporated into items such as bracelets, belts, back pads, mattress pads and mattresses. It is believed that static magnetic fields have some efficacy in the treatment of broken bones and soft tissue injuries, and tend to promote the circulation of blood as well as relieve stiffness in muscles. The effectiveness of such treatments in human and veterinary applications has been the subject of debate.

Resonant frequency therapy has been in use since the invention of electricity in the late 1800s. Pulsed electromagnetic fields (known as PEMF) were widely adopted in East and Western Europe but its use was restricted to animals in North America until. Veterinarians became the first health professionals to use PEMF therapy, usually to heal broken legs in racehorses, and by a scientist known as Royal Rife. Royal worked alongside Carl Zeiss in the microscope industry and conducted research in microscoping lighting frequencies. Using microscopes, he transmitted various frequency waves with a microscope device and targeted known pathogens. In tests using B. coli (bacillus coli) Rife found that the bacteria, if exposed to radio-frequency energy at certain frequencies, would “deactivate”, or die. Later testing showed the same results with other samples of B. Coli at the same frequencies. One of the original PEMF devices consisted of a Helmholtz coil which generated a magnetic field. The patient's body was placed inside the magnetic field to deliver treatment. In the 1950s, researchers began extrapolating these frequencies to treat human diseases and Dr. Abraham Ginsberg successfully treated patients who had various infections and went on to found the Ginsberg Foundation for Medical Research Symposium.

During the 1970s, a medical researcher named Bassett introduced a new approach for the treatment of delayed fractures. This technique employed a very low frequency signal to be applied for non-union, delayed fractures. The use of electrical stimulation in the lumbosacral region was first attempted by Alan Dwyer of Australia. In 1974, he reported successful initiation of graft incorporation in 11 of 12 fusion patients. In 2004, a pulsed electromagnetic field system was approved by the Food and Drug Administration (FDA) as an adjunct to cervical fusion surgery in patients at high risk for non-fusion. In 2009, a majority of PEMF wellness devices resembled a typical yoga mat in dimensions but were slightly thicker and housed several, flat, spiral coils to produce an even electromagnetic field. Frequency generators are often used to energize the coils to create a pulsed electromagnetic field. The majority of PEMFs was manufactured in Germany, Austria and Switzerland and was imported into North America as electric massagers or full body electric yoga mats. They were either placed on a massage table for clinical use or directly on the floor in the home to practice simple yoga postures. A wide variety of professional and consumer PEMF devices are sold and marketed as FDA registered wellness devices.

Electromagnetism has also been found to play a role in plant growth. In 1862, Louis Pasteur discovered that magnetism affected the growth of plants while experimenting with fermentation. He found that magnetism had a significant effect on the growth of plants and that such a treatment could be used effectively in agriculture by exposing seeds, water, soil and nutrients in the soil. This strategy is now used for agricultural development in many parts of the world. Later in the 1900s, researchers began experimenting with electromagnetism and seeds. U.S. Pat. No. 4,020,590 was issued to Albert R. Davis in for the apparatus and method of treating seeds with the magnetizer. The process of magnetizing seeds substantially accelerated the growth, and improved the quality of plants. The process of magnetizing water also has benefits to plants as it increases its solubility and filtering ability. Nutrients are dissolved easily in magnetized water, which can then be used for irrigation to increase the fertility of the soil. Magnetized water helps plants accumulate minerals in the water. This efficiency decreases the amount of water needed for irrigation. Fertilizers are also more easily dissolved in magnetized water and penetrate into the plant cell, thereby decreasing the amount of fertilizers required.

Electromagnetic stimulation during the fermentation of wines and alcoholic beverages has also had positive results. A French researcher named Matter found that continuously supplying low-power electromagnetic stimulation in the range of 20-30 kHz to S. cerevisiae (wine yeast) could enhance ethanol production. The optimum ultrasonic treatment at the frequency of 25 kHz resulted in 15.6% ethanol concentration that was higher than in the control system. Researchers at the University of Bologna have also demonstrated that the use of PEF can enhance the extraction of phenolic compounds in wine and increase color intensity in red wines. In fact, color stability was also assisted by the extraction of valuable polyphenolic compounds, monomers (flavonoids, hydroxycinnamic acids, tannins) and polymers (condensed tannins from grape skin) that occur during such exposure. The main mechanism responsible for the increasing mass transfer was the electroporation of vegetable cell membranes, which also enabled to reduce the maceration process, and to enhance the extraction of bioactive compounds from grape using PEMF.

In 2015, the FDA reclassified PEMF devices from the Class 3 category (insufficient safety information) to a Class 2 status (approved with safety measures). PEMF devices that have been FDA cleared to make health claims require a doctor's prescription for use. U.S. Pat. No. 5,402,782, issued Apr. 4, 1995, and U.S. Pat. No. 5,553,610, issued Sep. 10, 1996, both to Robert A. Lodder disclosed a small magnetic, near-infrared radiation and an acoustic wave delivery system for therapeutic purposes. U.S. Pat. No. 4,535,775 disclosed a method of healing bone fractures by applying to electrodes coupled to the skin of a living body in the vicinity of a bone fracture and transmitting an electromagnetic frequency in the range of 20-100 KHZ and a value in the range from about 2 to 10 volts. U.S. Pat. No. 9,812,246B1 granted to Nunez disclosed a toroid frequency coil, but it is not part of a larger treatment system. Several commercially available rife machines are available on the market that deliver electromagnetic frequencies using attachable electrodes, passive electrostatic tubes and hand held cylinders. However, none were found in the prior art that incorporated a coil bound in a multi-point pattern connected to external amplifiers and digital audio frequency files for mobile devices. In addition to the benefits of frequency coils, it is well-known that the addition of gem stones within such coils can have therapeutic benefits (known as piezoelectric or pyro-electric effects as additional ions are generated from the coil. Some of the more important benefits from ions are that they clear the air of airborne allergens such as pollen, mold spores, bacteria and viruses. Besides they also clear the air of dust, pet dander and cigarette smoke.

Several commercially available rife machines are available on the market that deliver electromagnetic frequencies using attachable electrodes, passive electrostatic tubes and hand held cylinders. However, none were found in the prior art that incorporated a coil bound in a multi-point pattern connected to external amplifiers and digital audio frequency files for mobile devices. In addition to the benefits of frequency coils, it is well-known that the addition of gem stones within such coils can have therapeutic benefits (known as piezoelectric or pyro-electric effects as additional ions are generated from the coil. Some of the more important benefits from ions are that they clear the air of airborne allergens such as pollen, mold spores, bacteria and viruses. Besides they also clear the air of dust, pet dander and cigarette smoke

More recent attempts to employ the therapeutic effects of magnetic fields have focused on devices which generate an electromagnetic field, and the methods of treatment employing such devices. Although a variety of designs have been proposed in the prior art, electromagnetic devices generally comprise a power supply coupled to a circuit capable of producing an AC or DC output which is transmitted to an inductor coil. One form of inductor coil consists of a number of wire windings wrapped about a coil body with an open or air center, or, alternatively, a ferrous core wrapped with wire windings. In response to the output from the circuit, an electromagnetic field is generated by the inductor coil which is then directed toward the area(s) of the body of a patient to be treated.

In many instances, the circuit of electromagnetic devices produces a pulsed or time-varying output in the shape of a square wave, sine wave, triangular wave or the like. Such output can be at essentially any selected frequency and voltage. A pulsed output from the circuit results in the production of a time-varying or pulsed magnetic field by the inductor coil. If the circuit emits an AC signal, the position of the north and south poles of the resulting magnetic field from the inductor coil changes with each cycle, whereas a DC output produces an electromagnetic field in which the position of the magnetic poles remains constant.

The application of the general concepts of the formation of electromagnetic fields noted above to the treatment of physical and mental disorders has resulted in a widely varying array of devices and treatment methods. There appears to be no consensus whatsoever as to what flux density levels or frequencies should be employed in electromagnetic therapy. Although proposed as a non-invasive alternative to pharmacological and nutritional solutions, it is believed that electromagnetic therapy conducted at the high flux density and/or high frequency levels noted above may, in fact, be harmful whereas treatment at the lower end of the spectrum as suggested in U.S. Pat. No. 6,099,459 will have little, if any, therapeutic effect without extensive technical expertise. None of these treatment methods are reflective of the magnetic field density levels and frequencies which occur naturally within a patient, or are produced naturally within the ionosphere and by the earth.

Other significant limitations of many prior art therapeutic electromagnetic devices is their lack of portability, their complexity and the need for relatively skilled medical personnel to operate them effectively. For example, U.S. Pat. Nos. 6,280,376; 6,099,459; 6,210,317 and application US 2002/0103411 disclose devices which are not portable and require a skilled technician or physician to operate. In order to receive treatment, patients must undertake the time and expense of traveling to the office where the machine is located during normal business hours. Other devices, while they may be more portable, permit a relatively wide range of adjustment of field strength and/or frequency. Allowing patients and practitioners to control these parameters, even with prior instruction, can lead to ineffective or potentially harmful treatment.

There remains a significant need for improved electromagnetic field generator system, methods, and technologies. Specifically, most prior efforts entail complex and difficult-to-use practices or systems. Some efforts are yet to simplify and improve the system focused on the electromagnetic fields which are not harmful to the users. Yet all efforts have failed to deliver the benefits. In addition, prior efforts have failed to describe electromagnetic field generator system, methods, and technologies generating electromagnetic fields from amplified, digital frequencies from a mobile computing device.

### SUMMARY OF THE INVENTION

The device (interchangeably hereinafter referred to as “apparatus” or “portable electromagnetic energy generating apparatus” or “portable electromagnetic field generator” or “invention”) herein disclosed and described provides a solution to the shortcomings in the prior art through the disclosure of a method and system for transmitting resonant frequencies through a portable copper coil emitter. The device can include a sound coupling with conductive poles arranged in a star-like, toroidal or cylindrical pattern. The poles are wound with copper wire and are connected to a digital audio frequency library on a mobile device. When activated, audio files are played into the amplifier where there frequencies are amplified and sent to the electromagnetic copper coil allowing it to emit an electromagnetic field.

It is therefore among the objectives of this invention to provide a method and apparatus for the treatment of physical and mental disorders with electromagnetic therapy which does not require skilled personnel to administer, which is portable, which operates at naturally occurring magnetic field strengths and frequencies, which limits the extent of operating adjustments permitted on the part of a patient or practitioner and which is capable of treating a wide variety of physical and mental disorders in human or animal subjects.

The present invention generally relates to resonant frequencies. More specifically, it relates to a method and system for generating electromagnetic fields from amplified, digital frequencies from a mobile computing device.

An object of the invention is to provide a means to deliver electromagnetic frequencies to specific areas of the user's body. The user plays an audio file on their mobile devices, and it sends the audio signal frequency to the copper coil and then spins electrons through the copper coil at the speed of light in a circular fashion creating a pulsed electromagnetic vortex field. If crystals or gems are attached to the copper coil, healthy ions are emitted by the crystals or gem. The user positions the unit within close proximity to an area of the body of concern.

Another object of the invention is to provide a means to allow a user adjust the level and of electromagnetic frequencies delivered. The user can use a setting on the software app to adjust the type and amount of frequencies that are emitted from the device depending on the therapy needed.

Another object of the invention is to provide a means to allow different types of coils to be mounted to the sound coupling. Users can select from a wide array of manufactured coils to use depending on the condition they would like to treat.

Another object of the invention is to provide a means operate the device wirelessly. Another embodiment has a receiver embedded into the sound coupling that allows the app on a mobile device to send audio signals to the coil and emit healing frequencies.

Another object of the invention is to allow the device to operate without a mobile device. An embodiment of the invention may include a powered base station that has digital audio files therein allowing a user to simply connect the device directly to it to generate the necessary healing frequencies.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

The summary of the invention does not necessarily disclose all the features essential for defining the invention. The invention may reside in a sub-combination of the disclosed features. The various combination and sub-combination are fully described in the detailed description.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

The diagrams are for illustration only, which thus is not a limitation of the present disclosure, and wherein:

FIG. 1 illustrates a perspective view of in accordance with an embodiment the invention.

FIG. 2 illustrates a perspective view of the invention being plugged into a phone.

FIG. 3 illustrates the invention being in operation in accordance with an embodiment the invention.

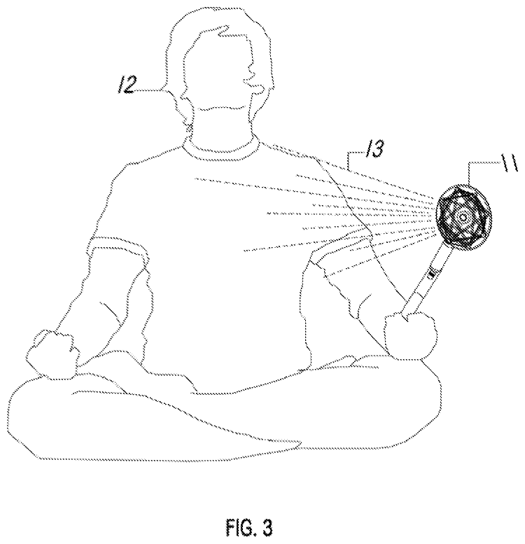

FIG. 4 illustrates a gem stone in accordance with an embodiment the invention.

FIG. 5 illustrates an embodiment with another type of coil.

FIG. 6 illustrates a wireless embodiment being worn by a user.

FIG. 7 illustrates an embodiment of the invention with a base station.

FIG. 8 illustrates the invention app user interface, in accordance with an embodiment the invention.

FIG. 9a-9d illustrates different views of the toroid, in accordance with an embodiment the invention.

FIG. 10 illustrates the cutout section showing 3 crystal chambers, in accordance with an embodiment the invention.

FIG. 11 illustrates the cutout section showing 3 crystal chambers according to another embodiment of the invention

FIG. 12 illustrates the cutout section showing 3 crystal chambers according to another embodiment of the invention

### DETAILED DESCRIPTION OF DRAWINGS

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure.

In the following description, numerous specific details are set forth in order to provide a thorough understanding of embodiments of the present invention. It will be apparent to one skilled in the art that embodiments of the present invention may be practiced without some of these specific details.

Various terms as used herein are shown below. To the extent a term used, it should be given the broadest definition persons in the pertinent art have given that term as reflected in printed publications and issued patents at the time of filing.

The present invention generally relates to resonant frequencies. More particularly, the present invention relates to a portable device capable of being operated safely and effectively by patients which produces a time varying, magnetic field having a low frequency and low flux density effective in the treatment of a wide variety of physical and mental disorders. More specifically, it relates to a method and system for generating electromagnetic fields from amplified, digital frequencies from a mobile computing device.

Embodiments of the present invention provide a portable electromagnetic field generator with interchangeable coil is disclosed. The invention includes three main parts. A first part being a sound coupling that can be ‘paired’ with a mobile computing device (such as a cell phone, tablet etc.); a second pan being an interchangeable, electromagnetic, resonant frequency coil (referred to herein as ‘coil’); and a mobile device software application (app) with an archive of digital therapy sound files that users can choose from. These therapy files are ported through said coils allowing users to receive a plurality of healing, electromagnetic fields and ions depending on their malady. Upon receiving sound file frequencies, said copper winding inside the coils conducting an electromagnetic field and emitting ions to be received by a user seeking therapeutic benefits. Other embodiments may incorporate a gem stone within the coil to enhance frequency effectiveness therein. An object of the invention is to provide a portable means to deliver an electromagnetic field frequencies normally associated with larger, table-based systems.

An embodiment of the present invention provides portable electromagnetic energy generating apparatus. The apparatus a include an electromagnetic energy generating means to produce one or more electromagnetic fields on receipt of at least an electric signal generated by one or more electric signal generating means, wherein the one or more produced electromagnetic fields are associated with the received electric signal, and wherein the one or more produced electromagnetic fields are adapted to provide a therapeutic effect to an area of a user body.

In an exemplary embodiment, the electromagnetic energy generating means comprise of a plurality electromagnetic copper coils.

In an exemplary embodiment, the plurality electromagnetic copper coils are arranged in any or a combination of a star-like, toroidal or cylindrical pattern. The electromagnetic energy generating means comprise of one or more conductive poles arranged in any or a combination of a star-like, toroidal or cylindrical patter, and wherein the one or more conductive poles are wound with one or more copper wires.

In an exemplary embodiment, the one or more electromagnetic fields include one or more ions

In an exemplary embodiment, the one or more electromagnetic fields is a pulsed electromagnetic vortex fields.

In an exemplary embodiment, the one or more electromagnetic fields is generated at least at variable frequencies or at non-variable frequencies.

In an exemplary embodiment, the apparatus can further includes a connecting means for connecting the portable electromagnetic energy generating apparatus with the one or more electric signal generating means to receive the electric signal from the one or more electric signal generating means at least in a wired communication mode or in a wireless communication mode.

In an exemplary embodiment, the apparatus can further includes at least a crystal or a gem to generate one or more ions in presence of the one or more produced electromagnetic fields.

In an exemplary embodiment, the apparatus can further includes an adjuster means to adjust the one or more produced electromagnetic fields.

In an exemplary embodiment, the one or more electric signal generating means is a mobile application installed in an electronic device.

In an exemplary embodiment, the electric signal comprising an audio signal.

In an exemplary embodiment, the apparatus is a wearable device.

Another embodiment of the present invention provides a portable electromagnetic energy generating apparatus. The apparatus includes a connecting means for connecting the portable electromagnetic energy generating apparatus with one or more electric signal generating means to receive the electric signal from the one or more electric signal generating means at least in a wired communication mode or in a wireless communication mode; and an electromagnetic energy generating means to produce one or more electromagnetic fields on receipt of the electric signal generated by the one or more electric signal generating means, wherein the one or more produced electromagnetic fields are associated with the received electric signal.

In an exemplary embodiment, the electromagnetic energy generating means comprise of a plurality electromagnetic copper coils, arranged in any or a combination of a star-like, toroidal or cylindrical pattern.

In an exemplary embodiment, the electromagnetic energy generating means comprise of one or more conductive poles arranged in any or a combination of a star-like, toroidal or cylindrical patter, and wherein the one or more conductive poles are wound with one or more copper wires.

In an exemplary embodiment, the one or more electromagnetic fields are pulsed electromagnetic vortex fields.

In an exemplary embodiment, the one or more electromagnetic fields are generated at least at variable frequencies or at non-variable frequencies.

In an exemplary embodiment, the apparatus includes at least a crystal or a gem to generate one or more ions in presence of the one or more produced electromagnetic fields.

Another embodiment of the present invention provides a portable electromagnetic energy generating apparatus. The apparatus includes a connecting means for connecting the portable electromagnetic energy generating apparatus with one or more electric signal generating means to receive the electric signal from the one or more electric signal generating means at least in a wired communication mode or in a wireless communication mode; an electromagnetic energy generating means to produce one or more electromagnetic fields on receipt of the electric signal generated by the one or more electric signal generating means, wherein the one or more produced electromagnetic fields are associated with the received electric signal; and at least a crystal or a gem to generate one or more ions in presence of the one or more produced electromagnetic fields.

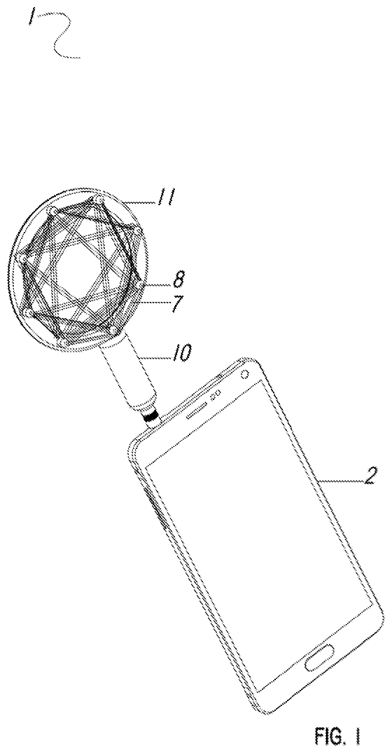

FIG. 1 illustrates a perspective view of a plugin embodiment of the apparatus 1 that connects to a cell/mobile phone 2. In an exemplary embodiment FIG. 1 illustrates a wheel support 11 connecting a metal wire coil 7 connected by means of circular-shaped bolts 8. The wire 7 can include, but is not limited to, a conductive metal such as, but not limited to copper etc. and said bolts 8 made of a rigid metal such as stainless steel aluminum, steel and the like. The bolts 8 being disposed radially at, but not limited to a plurality of points around said wheel support 11.

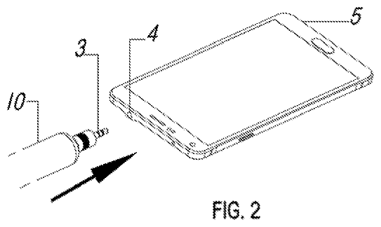

FIG. 2 illustrates a perspective view of the invention being plugged into a phone. Metal wire coil 7 (as shown in FIG. 1) having at least three levels of windings and connected to audio devices by means of an audio jack 3 therein. A distal end of the invention having a cylinder-shaped sound coupling 10 containing terminal ends of said wires 7 and being made of a rigid material such as plastic and the like.

In an exemplary embodiment FIG. 2 illustrates a perspective view of the sound coupling 10 with conventional audio jack 3 being configured to be received by an audio port 4 in mobile device 5 and allowing digital audio sound files to be transmitted to coil 7. The invention may be plugged into a multitude of audio ports on devices such as but not limited to: cell phones, tablets, desktop computers and the like.

In an exemplary embodiment FIG. 1 illustrates the apparatus 1 in use with its electromagnetic field 13 being emitted from the metal wire coil 7 (as shown in FIG. 1) and rotating a signal at the speed of light in a circular fashion creating a frequency pulsed electromagnetic field with a plurality of positive ions that surrounds a user 12.

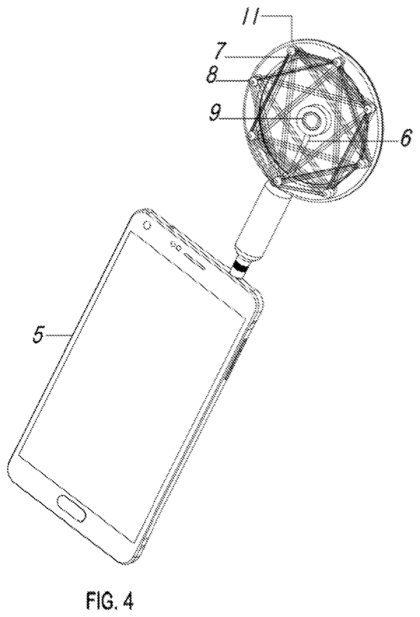

In an exemplary embodiment FIG. 4 illustrates another embodiment of the apparatus 1 having a stone 9, including but not limited to gems, sapphires, rubies, quartz and the like being disposed proximally on support wheel support 11 by means of cross member 6 there between.

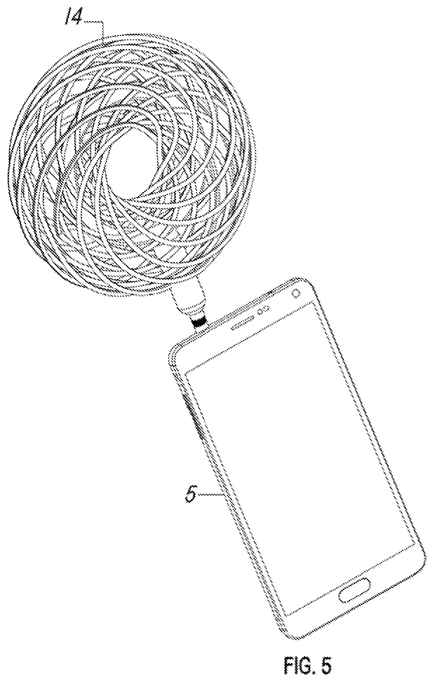

In an exemplary embodiment FIG. 5 illustrates a perspective view of the plugin embodiment of the apparatus 1 with a toroid-shaped coil 14. The apparatus 1 having the sound coupling 10 (as shown in FIG. 1) configured to receive a plurality of coils such as but not limited to Qi Coils™, POE coils, toroidal nested coils and the like.

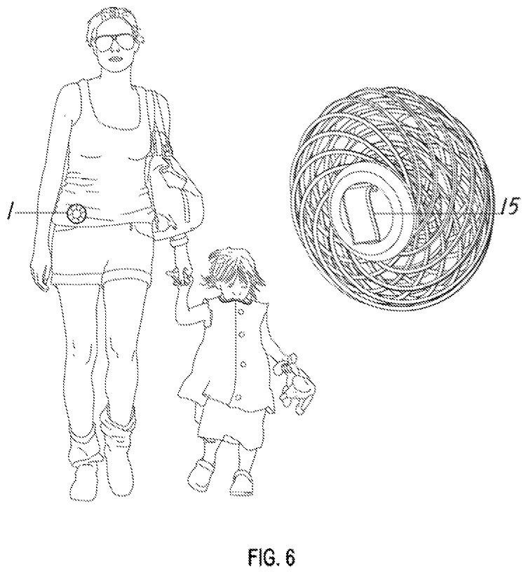

In an exemplary embodiment FIG. 6 illustrates a user wearing the wireless embodiment of the apparatus 1 on their persons by means of clip 15. In this embodiment of invention the apparatus 1 can include a wireless receiver (not shown) to retrieve app digital audio signals from mobile device (not shown) by means of BlueTooth™ therein.

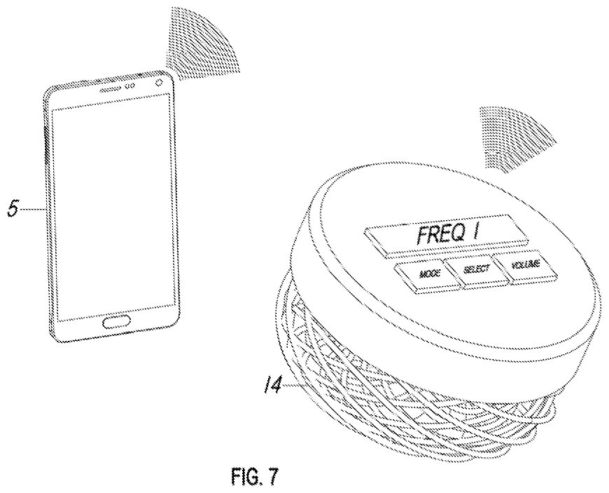

In an exemplary embodiment FIG. 7 illustrates a standalone base station (shown as a mobile phone 5) embodiment of the invention sending audio content to the device without the need for a user's mobile computing device. The base station can include a proprietary digital audio player configured to perform a varietal of functions such as but not limited to selecting the digital file and controlling digital file intensity, volume, frequency, tone and the like. The base station also having software memory to hold a multitude of proprietary digital audio files therein as well as a small user interface screen.

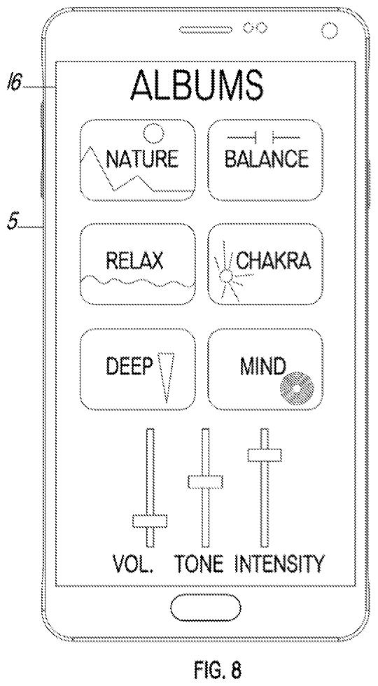

In an exemplary embodiment FIG. 8 illustrates a mobile application 16 being operated on a mobile device 5 and having sound file selections that allow users to determine which types of audio frequencies are transmitted to coil 7 (as shown in FIG. 1). Said audio files include a plurality of ‘Life Force’ frequencies associated known to be associated with mitigating a multitude of maladies such as, but not limited to: Allergies, emotional issues, chronic illnesses etc. Frequencies may include but not be limited to: 1 Hz to 30,000 Hz at a multitude of incremental frequencies preset and/or harmonic frequencies that play multiple frequencies at the same time by said digital audio devices. Said app 16 having a multitude of setting options such as but not limited to audio volume, tone and intensity etc. Some embodiments of said app 16 may be purchased by users as part of a subscription and compatible with existing app services including but not limited to Google Play, Apple etc.

In an exemplary embodiment FIG. 8 further illustrates that the software application can include functions such as but not limited to varietal of functions such as but not limited to selecting the digital file, controlling digital file intensity, volume, frequency, tone and the like. Said invention software being written from code that may include, but not be limited to: Java, C++™, Visual Basic™, Fortran™, Basic™ and the like. The software also compatible with a plurality of operating systems such as, but not limited to: Windows™, Apple™, and Android™, and compatible with a multitude of hardware platforms such as, but not limited to: personal desktops, laptops, tablets, smartphones and the like.

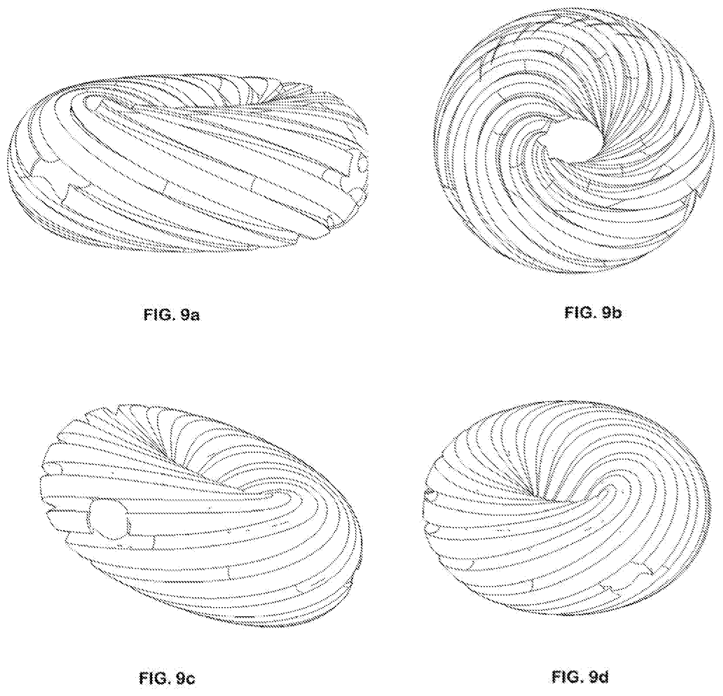

In an exemplary embodiment FIG. 9a-9d, illustrates multiple view of the plugin embodiment of the apparatus 1 with a toroid-shaped coil. The multiple views illustrate various sides of the toroid having one or more openings for receiving plug or terminals for connecting to other devices.

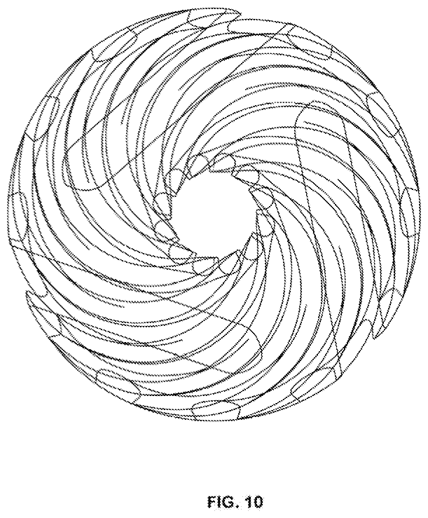

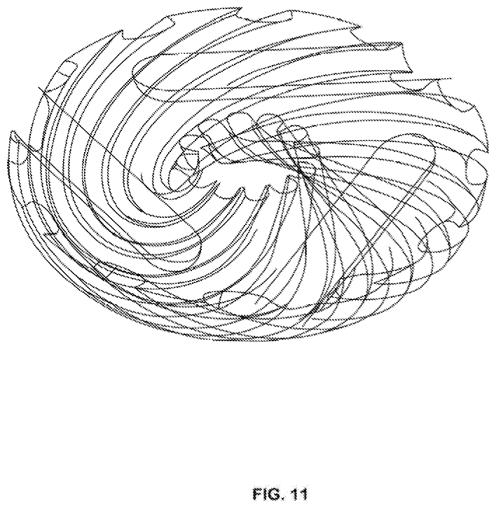

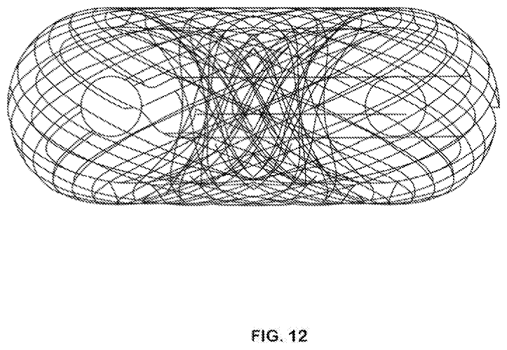

In an exemplary embodiment FIGS. 10, 11 and 12, illustrates a toroid-shaped coil. The cutout section of the toroid shaped coil illustrates toroid having one or more openings for receiving plug or terminals for connecting to other devices.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware and/or by human operators.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention.

What is claimed is:

1. A portable electromagnetic energy generating apparatus, comprising:
an electromagnetic energy generating means to produce one or more electromagnetic fields on receipt of at least an electric signal generated by one or more electric signal generating means, wherein the one or more produced electromagnetic fields are associated with the received electric signal, and wherein the one or more produced electromagnetic fields are adapted to provide a therapeutic effect to an area of a user body.

2. The portable electromagnetic energy generating apparatus according to claim 1, wherein the electromagnetic energy generating means comprise of a plurality electromagnetic copper coils.

3. The portable electromagnetic energy generating apparatus according to claim 2, wherein the plurality electromagnetic copper coils are arranged in any or a combination of a star-like, toroidal or cylindrical pattern.

4. The portable electromagnetic energy generating apparatus according to claim 1, wherein the electromagnetic energy generating means comprise of one or more conductive poles arranged in any or a combination of a star-like, toroidal or cylindrical patter, and wherein the one or more conductive poles are wound with one or more copper wires.

5. The portable electromagnetic energy generating apparatus according to claim 1, wherein the one or more electromagnetic fields include one or more ions.

6. The portable electromagnetic energy generating apparatus according to claim 1, wherein the one or more electromagnetic fields are a pulsed electromagnetic vortex fields.

7. The portable electromagnetic energy generating apparatus according to claim 1, wherein the one or more electromagnetic fields are generated at least at variable frequencies or at non-variable frequencies.

8. The portable electromagnetic energy generating apparatus according to claim 1, further comprising: a connecting means for connecting the portable electromagnetic energy generating apparatus with the one or more electric signal generating means to receive the electric signal from the one or more electric signal generating means at least in a wired communication mode or in a wireless communication mode.

9. The portable electromagnetic energy generating apparatus according to claim 1, further comprising: at least a crystal or a gem to generate one or more ions in presence of the one or more produced electromagnetic fields.

10. The portable electromagnetic energy generating apparatus according to claim 1, further comprising: an adjuster means to adjust the one or more produced electromagnetic fields.

11. The portable electromagnetic energy generating apparatus according to claim 1, wherein the one or more electric signal generating means is a mobile application installed in an electronic device.

12. The portable electromagnetic energy generating apparatus according to claim 1, wherein the electric signal comprising an audio signal.

13. The portable electromagnetic energy generating apparatus according to claim 1, wherein the apparatus is a wearable device.

14. A portable electromagnetic energy generating apparatus, comprising:
a connecting means for connecting the portable electromagnetic energy generating apparatus with one or more electric signal generating means to receive the electric signal from the one or more electric signal generating means at least in a wired communication mode or in a wireless communication mode;
an electromagnetic energy generating means to produce one or more electromagnetic fields on receipt of the electric signal generated by the one or more electric signal generating means, wherein the one or more produced electromagnetic fields are associated with the received electric signal.

15. The portable electromagnetic energy generating apparatus according to claim 13, wherein the electromagnetic energy generating means comprise of a plurality electromagnetic copper coils, arranged in any or a combination of a star-like, toroidal or cylindrical pattern.

16. The portable electromagnetic energy generating apparatus according to claim 13, wherein the electromagnetic energy generating means comprise of one or more conductive poles arranged in any or a combination of a star-like, toroidal or cylindrical patter, and wherein the one or more conductive poles are wound with one or more copper wires.

17. The portable electromagnetic energy generating apparatus according to claim 13, wherein the one or more electromagnetic fields are a pulsed electromagnetic vortex fields.

18. The portable electromagnetic energy generating apparatus according to claim 13, wherein the one or more electromagnetic fields are generated at least at variable frequencies or at non-variable frequencies.

19. The portable electromagnetic energy generating apparatus according to claim 13, further comprising: at least a crystal or a gem to generate one or more ions in presence of the one or more produced electromagnetic fields.

20. A portable electromagnetic energy generating apparatus, comprising:
a connecting means for connecting the portable electromagnetic energy generating apparatus with one or more electric signal generating means to receive the electric signal from the one or more electric signal generating means at least in a wired communication mode or in a wireless communication mode;
an electromagnetic energy generating means to produce one or more electromagnetic fields on receipt of the electric signal generated by the one or more electric signal generating means, wherein the one or more produced electromagnetic fields are associated with the received electric signal; and
at least a crystal or a gem to generate one or more ions in presence of the one or more produced electromagnetic fields.
